(19) United States
(12) Patent Application Publication
(10) Pub. No.: US 2026/0182954 A1
(43) Pub. Date: Jul. 2, 2026

(54) ULTRASOUND MEASURING SYSTEMS AND METHODS WITH TIME DOMAIN REFLECTOMETRY

(71) Applicant: Provisio Medical, Inc., San Diego, CA (US)

(72) Inventors: Stephen Eric Ryan, San Diego, CA (US); Randy-Alexander Randolph Besprozvanny, San Diego, CA (US)

(21) Appl. No.: 19/129,235

(22) PCT Filed: Nov. 17, 2023
(86) PCT No.: PCT/US2023/080367

§ 371 Date: May 12, 2025

Related U.S. Application Data
(60) Provisional application No. 63/384,752, filed on Nov. 22, 2022.

(51) Int. Cl. A61B 8/12; A61B 8/00; A61B 8/08; G06T 7/50; G06T 7/73

(52) U.S. Cl. CPC A61B 8/12; A61B 8/0891; A61B 8/5223; G06T 7/50; G06T 7/73; G06T 2207/10132; G06T 2207/30101

(57) ABSTRACT

An ultrasound system for measuring the dimensions of a structure, the system including a flexible body elongated along a longitudinal axis for insertion into the structure. At least one ultrasound transducer arranged on the flexible body and configured to transmit and receive ultrasound signals to and from the structure. The system is configured to calculate distances between each transducer and the structure based on the received ultrasound signals and to calculate multiple cross-sectional shapes of the structure based on the distances. The flexible body includes one or more electrical waveguides. The system causes electrical pulses to travel through the one or more wave-guides and a conductive element through which the flexible body moves to make time domain reflectometry (TDR) distance measurements based on responsive impedance signals created by the conductive element. The system is configured so that the TDR distance measurements correspond to relative longitudinal positions of the flexible body.

### REFERENCE TO RELATED APPLICATIONS

This application claims the benefit of U.S. Provisional Application No. 63/384752, filed Nov. 22, 2022, which is hereby incorporated by reference in its entirety.

### BACKGROUND

### Field of the Disclosure

The present disclosure relates generally to systems, methods, and devices that utilize ultrasound to gather dimensional and physiological information about structures such as fluid-filled body vessels.

### Description of Related Art

Obtaining and utilizing structural information about patients is a critical aspect of diagnosing and treating many medical conditions. For example, within the field of endovascular medicine, it is important to gain structural and physiological information about diseased blood vessels when selecting among interventional techniques such as angioplasty, stents, and/or surgery. Recent studies have illustrated that the predominate cause of endovascular treatment failure is inaccurate sizing of vessels or inadequate treatment to achieve the lumen dimensions desired over an entire stenotic lesion. An improperly selected, dimensioned, and/or positioned medical device (e.g., a stent) and/or treatment can lead to highly adverse outcomes including avoidable death. Typical techniques used for analyzing the structural features of blood vessels include angiography. However, angiography only provides limited and imprecise information about the size and morphology of blood vessels and often does not allow the physician to adequately assess the lesion prior to treatment. Recent studies have shown that outcomes are significantly improved through the use of more advanced, more accurate imaging techniques.

Some imaging catheters utilize ultrasound or optical technologies to provide a more accurate cross-sectional imaging that may then be interpreted by the physician to determine, among other characteristics, the dimensions of the lumen surrounding the catheter. For example, Intravascular Ultrasound (IVUS) and Optical Coherence Tomography (OCT) have been used in interventional diagnostic procedures to image blood vessels to locate and characterize atherosclerosis and other vessel diseases and defects.

IVUS and OCT images can be used to determine information about a vessel, including vessel dimensions, and is typically much more detailed than the information that is obtainable from traditional angiography images, which are generally limited to two-dimensional shadow images of the vessel lumen. The information gained from more accurate imaging techniques can be used to better assess physiological conditions, select particular procedures, and/or improve performance of the procedure.

While current IVUS and OCT systems provide additional and more detailed information compared to angiograms, these IVUS and OCT systems introduce significant additional time, cost and complexity into minimally-invasive procedures. The components of these systems (e.g., transducers, wires, imaging circuitry, fiber-optics, etc.) can occupy a large footprint within the blood vessel and must often be deployed independently and at separate times from interventional procedures (e.g., angioplasty). Adding to these components and their footprints are those used to guide and track the location of imaging/treatment catheters within a blood vessel so as to be able to effectively guide treatment. These components often include imprecise mechanical systems and/or imprecise angiography imaging. Subsequently, the images produced by IVUS and OCT systems may not directly provide useful information about blood vessels and are typically subject to nonconforming interpretations of different physicians. Thus, there is a need for an improved and more efficient way to get needed information about a vessel or structure, particularly information about the diameter, multi-dimensional profile, and precise location of a vessel or structure, while not sacrificing speed and footprint needed for timely, efficient, and effective treatment.

### SUMMARY

Embodiments of the present disclosure include a novel implementation of an ultrasound measurement probe to approximate the dimensions and/or shape(s) of fluid-filled structures. Some embodiments include an elongated flexible body such as a catheter with multiple ultrasound transducers arranged circumferentially about the catheter for generating and receiving ultrasound signals to and from surrounding structure. One or more electrically conductive waveguides (e.g., wires, cables) extend from a base of the flexible probe to a position along the flexible probe distal to its base and are connected with a time domain reflectometry (TDR) sensor.

A computing system is connected with the TDR sensor and ultrasound transducers. The computing system is programmed and configured to obtain signals responsive to the transmitted ultrasound signals (pulses) and to calculate a distance measurement between the ultrasound probe and the structure based on each of the responsive signals. The distance measurements may be used to determine points of a wall boundary (e.g., vessel wall) of the structure and a cross-section of the structure based on a curve-fit to the points.

The TDR sensor is used to transmit separate electrical pulses through the one or more conductive waveguides and obtain responsive TDR signals. The TDR signals are used to detect a change in impedance along the conductive waveguides and the computing system is used to calculate a distance along the probe between the base of the probe and the source of change in impedance. In some embodiments, the conductive waveguides are manufactured out of polyimide or liquid crystal polymer. In some embodiments, the electrical pulses are delivered at frequencies of between about 5 to 10 MHz.

In some embodiments, a conductive element is used as the source of the detected impedance. The conductive element may be positioned on the probe and remain stationary while the probe longitudinally moves. The conductive element is arranged to be in contact (e.g. short circuited) with the conductive waveguides and to impede electronic transmissions through the waveguides. The conductive element may be a conductive ring surrounding the probe body and is arranged so that the probe body moves within the ring as the probe body is longitudinally actuated (e.g., inserted or withdrawn from a structure such as a blood vessel). As the probe body moves within the ring in response to actuating the probe body, a determination is made of the relative longitudinal position of the probe within the structure (e.g., within a blood vessel). In some embodiments, a conductive medium (e.g., blood) in which the ultrasound probe enters a structure is utilized as the source of detected impedance.

In some embodiments, a single waveguide is used to obtain responsive TDR signals. When an electrical pulse comes into contact with a conductive element some of the energy from the pulse is reflected back along the waveguide. The time it takes this reflected energy to travel the length of the waveguide to the conductive element and back can be used to determine longitudinal positions of the catheter.

In some embodiments, more than one waveguide can be used to obtain TDR signals. An electrical pulse transmitted through one waveguide until it comes into contact with the conductive element. The signal would be transmitted to another waveguide through the conductive element. As the longitudinal position of the catheter changes the length of the circuit created by the waveguides and conductive element would also change. The time it takes the transmitted energy to travel the length of the circuit may be used to determine the longitudinal positions of the catheter.

Using the TDR-calculated longitudinal positions of the probe and the ultrasound-generated cross-sections of the structure, the cross-sections are registered with respect to each other by the computing system in order to generate a longitudinal profile/map of the structure. The longitudinal profile/map may be represented as a three-dimensional profile of the structure by fitting/interpolating the areas between cross-sections to each other based on their longitudinal positions.

For purposes of summarizing the disclosure and the advantages achieved over the prior art, certain objects and advantages of the disclosure are described herein. Not all such objects or advantages may be achieved in any particular embodiment. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, a method of ultrasounds measuring is described herein. The method includes longitudinally actuating an ultrasound probe between a plurality of positions within the structure. While the ultrasound probe is at each of the plurality of positions, the method may further include transmitting ultrasound signals from at least one ultrasound transducer of the ultrasound probe toward a structure, obtaining signals responsive to the transmitted ultrasound signals and calculating distance measurements between the ultrasound probe and the structure based on each of the responsive signals, transmitting electrical pulses through one or more conductive waveguides extending between a proximate end of the ultrasound probe and a portion of the probe distal to the proximate end, obtaining reflective signals responsive to the electrical pulses, the reflective signals representing a change in characteristics of the transmitted electrical pulses as they transmit through a particular longitudinal position of the one or more conductive waveguides, and calculating one or more longitudinal position measurements of the ultrasound probe based on the reflective signals responsive to the electrical pulses.

In some embodiments, calculating the longitudinal position measurements is based on analyzing time domain reflectometry (TDR) waveforms within the reflective signals. In some embodiments, the TDR waveforms are generated in response to an impedance change through the one or more conductive waveguides created by a conductive element positioned along the ultrasound probe, wherein the conductive element is arranged to remain stationary as the ultrasound probe is longitudinally actuated. In some embodiments, the conductive element includes a movably slidable ring arranged about the ultrasound probe. In some embodiments, the conductive element is integrated within a trocar through which the ultrasound probe is arranged to pass, the trocar configured with a mechanism for locking the trocar in place to the probe. In some embodiments, the conductive element includes a fluid media in the structure within which the ultrasound probe is longitudinally actuated. In some embodiments, the fluid media includes blood. In some embodiments, calculating the longitudinal position measurements is based on electrical pulses transmitted through one conductive waveguide. In some embodiments, calculating the longitudinal position measurements is based on analyzing time domain reflectometry (TDR) waveforms within radio frequency waves. In some embodiments, the electrical pulses comprise a wavelength range of between about ten and fifteen megahertz. In some embodiments, a single waveguide is used and reflected energy is analyzed. In some embodiments, multiple waveguides are used and transmitted energy is analyzed. In some embodiments the method includes determining a plurality of cross-sectional shapes of the structure based on the calculated distance measurements between the ultrasound probe and the structure. In some embodiments, the method includes determining a longitudinal distance between the cross-sectional shapes based on the one or more longitudinal distance measurements. In some embodiments, the method includes determining one or more longitudinal shapes of the structure based on the plurality of cross-sectional shapes and one or more longitudinal distances. In some embodiments, the method includes determining a three-dimensional shape of the structure based on the determined longitudinal shapes and the plurality of cross-sectional shapes. In some embodiments, the plurality of cross-sectional shapes are registered in computer memory as relative longitudinal positions within the structure based on the one or more longitudinal measurements.

In another aspect, an ultrasound system for measuring dimensions of a structure is provided. The system includes a flexible body elongated along a longitudinal axis and assembled for insertion into the structure. The system further includes at least one ultrasound transducer arranged on the flexible body, one or more conductive waveguides extending between a proximate end of the flexible body and a portion of the flexible body distal to the proximate end, one or more processors programmed and configured to transmit ultrasound signals from the at least one ultrasound transducer, obtain signals responsive to the transmitted ultrasound signals and calculate a distance measurement between the flexible body and the structure based on each of the responsive signals, transmit electrical pulses through the one or more conductive waveguides, obtain reflective signals responsive to the electrical pulses, the reflective signals representing a change in characteristics of the transmitted electrical pulses as they transmit through a particular longitudinal position of the one or more conductive waveguides, and calculate one or more longitudinal position measurements of the flexible body based on the reflective signals responsive to the electrical pulses.

### BRIEF DESCRIPTION OF THE DRAWINGS

Embodiments of the disclosure will be described hereafter in detail with particular reference to the drawings. Throughout this description, like elements, in whatever embodiment described, refer to common elements wherever referred to and reference by the same reference number. The characteristics, attributes, functions, interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All figures are drawn for ease of explanation of the basic teachings of the present disclosure only; the extensions of the figures with respect to number, position, relationship and dimensions of the parts to form examples of the various embodiments will be explained or will be within the skill of the art after the present disclosure has been read and understood.

FIG. 1 is an illustrative diagram of an ultrasound catheter probe system according to some embodiments.

FIG. 2A is an illustrative side perspective diagram of an ultrasound catheter probe placed within a lumen at different positions according to some embodiments.

FIG. 2B is a cross-sectional perspective diagram of the ultrasound catheter probe of FIG. 2A.

FIG. 3A, is an illustrative side perspective view of a probe body integrated with TDR waveguides according to some embodiments.

FIG. 3B is an illustrative chart of TDR signals detected from the TDR waveguide of FIG. 3A according to some embodiments.

FIG. 3C is an illustrative side perspective view of a probe body integrated with TDR waveguide according to some embodiments.

FIG. 3D is an illustrative chart of TDR signals detected from the TDR waveguide of FIG. 3C according to some embodiments.

FIG. 3E is an illustrative side perspective view of a probe body integrated with TDR waveguide according to some embodiments.

FIG. 3F is an illustrative chart of TDR signals detected from the TDR waveguide of FIG. 3E according to some embodiments.

FIG. 4A is an illustrative cross-sectional view of a flat cable for use with a TDR system and an ultrasound probe according to some embodiments.

FIG. 4B is an illustrative perspective view of different types of waveguide arrangements for use with a TDR system and an ultrasound probe according to some embodiments.

FIG. 4C is an illustrative perspective view of a probe segment utilizing a conductive fluid medium as an impedance element for TDR implementation according to some embodiments.

FIG. 5A is an illustrative diagram of a TDR open circuit arrangement and chart of a TDR signal.

FIG. 5B is an illustrative diagram of a TDR parallel impedance arrangement and chart of a TDR signal according to some embodiments.

FIG. 5C is an illustrative diagram of a TDR short circuit arrangement and chart of a TDR signal according to some embodiments.

FIG. 5D is an illustrative diagram of a single-cable TDR arrangement presented with different types of impedance and a chart of corresponding TDR signals according to some embodiments.

FIG. 6 is an illustrative view of a TDR system integrated with a probe according to some embodiments.

FIG. 7 is an illustrative view of an ultrasound probe with a TDR sensing system inserted into a heart according to some embodiments.

FIG. 8 is an illustrative flow chart of a process for performing ultrasound measurements with longitudinal position measurements according to some embodiments.

FIG. 9A is an illustrative side perspective view of an ultrasound measurement probe longitudinally actuated within a vessel structure according to some embodiments.

FIG. 9B is an illustrative mapping of the vessel structure of FIG. 9A based on ultrasound and TDR measurements obtained according to some embodiments.

### DETAILED DESCRIPTION OF THE INVENTION

In order that embodiments of the disclosure may be clearly understood and readily carried into effect, certain embodiments of the disclosure will now be described in further detail with reference to the accompanying drawings. The description of these embodiments is given by way of example only and not to limit the scope of the disclosure.

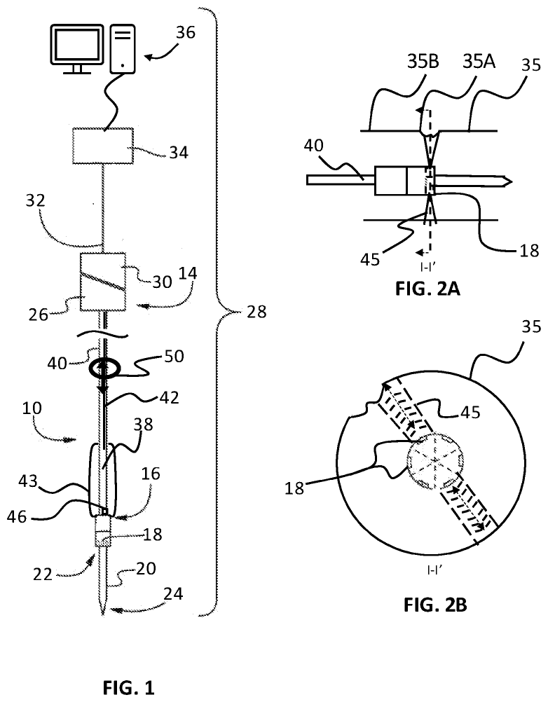

FIG. 1 is an illustrative diagram of an ultrasound catheter probe system 28 according to some embodiments. An ultrasound imaging probe 10 includes a probe body 40 having a proximal end 14 and a distal end 16. The probe 10 includes a plurality of transducers 18. In some embodiments, probe 10 includes an elongated tip 20 with a proximal end 22 and a distal end 24. Probe 10 includes a proximal connector 26 which connects probe 10 to other components of system 28, including a computer system 36. In an embodiment of the invention, the medical device 10 is part of a system 28 that includes a distal connector 30, electrical conductors 32, a data acquisition unit 34 and a computer system 36.

In some embodiments, probe body 40 is tubular and has a central lumen 38. In some embodiments, probe body 40 has a diameter of about 650 μm or less. These dimensions are illustrative and not intended to be limiting. In some embodiments, the diameter of the probe 10 will depend on the type of device that probe 10 is integrated with and where the probe 10 will be used (e.g., in a blood vessel), which will become apparent to those of ordinary skill in the art in view of the present disclosure.

The proximal end 14 of the probe body 40 is attached to the proximal connector 26. Elongated tip 20 has its proximal end 22 attached to the distal end 16 of probe body 40. The probe body 40 and elongated tip 20 may be constructed with an appropriate size, strength, and flexibility to be used for guiding probe 10 through a body lumen (e.g., a blood vessel). Elongated tip 20 and/or other components of probe 10 may include a radio-marker (e.g., visible to angiography) for precisely guiding the catheter through a lumen and positioning transducers 18 in the desired location. In some embodiments, probe 10 and distal end 16 are constructed and arranged for rapid exchange use. Probe body 40 and elongated tip 20 may be made of resilient flexible biocompatible material such as is common for IVUS and intravascular catheters known to those of ordinary skill in the art. In some embodiments, probe 10 includes a therapy-delivering device 43 such as an angioplasty balloon.

Probe body 40 has a tubular body with a central lumen 38. In some embodiments, probe 10 may have lumens for use with various features not shown (guidewires, fiberoptics, saline flush lumens, electrical connectors, etc.). In some embodiments, the outer diameter of the probe body 40 and elongated tip 20, if present, is substantially consistent along its length and does not exceed a predetermined amount.

In some embodiments, ultrasound transducers 18 are piezoelectric. The transducers may be built using piezoelectric ceramic or crystal material, or composites of piezoelectric ceramic or crystal with polymers, and layered by one or more matching layers that can be thin layers of epoxy, epoxy composites/mixtures, or polymers. In some embodiments, the transducers are PMUTs (Piezoelectric Micromachined Ultrasonic Transducers), CMUTs (Capacitive Micromachined Ultrasonic Transducers), and/or photoacoustic transducers.

The operating frequency for the ultrasound transducers may be in the range of from about 8 MHz to about 50 MHz or even up to about 60 MHz, depending on the dimensions and characteristics of the transducer and requirements of the particular application. Generally, higher frequency of operation provides better resolution and a smaller medical device 10. However, the tradeoff for this higher resolution and smaller catheter size may be a reduced depth of penetration into the tissue of interest and increased echoes from the blood itself (making the image more difficult to interpret). Lower frequency of operation is more suitable for imaging in larger vessels or within structures such as the chambers of the heart. Although specific frequency ranges have been given, these ranges given are illustrative and not limiting. The ultrasonic transducers 18 may produce and receive any frequency that leaves a transducer 18, impinges on some structure or material of interest and is reflected back to and picked up by a transducer 18.

The center resonant frequency and bandwidth of a transducer is generally related to the thickness of transducer materials generating or responding to ultrasound signals. For example, in some embodiments, a transducer includes a piezoelectric material such as quartz and/or lead-zirconate-titanate (PZT). A thicker layer will generally respond to a longer wavelength and lower frequency and vice versa. For example, a 50-micron thick layer of PZT will have a resonant frequency of about 40 MHz, a 65-micron thick layer will have a resonant frequency of about 30 MHz, and a 100-micron layer will have a resonant frequency of about 20 MHz. As further described herein, matching and backing layers may be included which affect the bandwidth and other characteristics of a transducer.

In some embodiments, probe 10 is connected with an actuating mechanism that may rotate and/or longitudinally move at least some portions of probe 10 and its transducers 18. A controlled longitudinal and/or radial movement permits the probe to obtain ultrasound readings from different perspectives within a surrounding structure, for example. Positioning the probe and its transducers in target locations may be augmented/guided by real-time imaging feedback provided by the transducers and system 28. Relative positions of the probe may be tracked and recorded during such processes (e.g., by using an encoder or other position sensing tool).

In some embodiments, system 28 is programmed to analyze and identify characteristics of the medium (e.g., blood) between probe 10 and structure in order to determine where the medium ends with respect to the structure (e.g., blood vessel wall). In some embodiments, multiple ultrasound measurements of the blood may be generated and the differences between the measurements are used to identify movement/change of the blood over time (e.g., as a result of a heart pumping). In some embodiments, doppler echo signals are used to determine these differences. Because the blood vessel wall does not have the same movement/change characteristics as the blood, the amount (or distance) between the probe 10 and blood vessel wall can be calculated. In some cases, reliance on the blood measurements without substantial reliance on measurements of the blood vessel wall may be used to determine the distance between probe 10 and blood vessel wall.

Computer system 36 is programmed to analyze and distinguish between the echoes associated with respective pulses. The computer system 36 is programmed to analyze the signals and calculate a radial distance measurement (e.g. D1, D2, . . . , D6) between each transducer 18 and lumen 35. This may be done, for example, by utilizing time-of-flight information of the echo signals and previously determined/differentiated signatures representative of a lumen wall (e.g., of lumen 35) and a particular medium (e.g., blood) between the transducer and lumen 35. Exemplary systems and methods for making such calculations are described, for example, in U.S. Pat. No. 10,231,701 filed Mar. 14, 2014 (the '701 Patent), the entire contents of which is herein incorporated by reference.

Based on distance calculations (D1, D2, . . . , D6), the shape and dimensions of lumen 35 may be estimated by further utilizing information including the dimensions of probe 10 and applying interpolation and/or other mathematical fitting techniques. For example, the relative positions of points (p1, . . . , p6) about lumen 35 may first be calculated and a curve fitting algorithm (e.g., spline interpolation) is applied to generate a two-dimensional slice representation of the lumen 35. As described in the '701 Patent, multiple slices can be calculated by taking sets ultrasound readings along the longitudinal extent of lumen 35 and combining them to generate a three-dimensional representation. In some embodiments, one or more transducers 46 are positioned within balloon 43 and are used to calculate the level of expansion of balloon 43 as it is expanded, for example.

Probe body 40 includes one or more electrical waveguides 42 extending between a proximal connector 26 and a position along probe segment 40 that is distal to a conductive impedance element 50. The electrical waveguides 42 are connected to a TDR sensor integrated into acquisition device 36 connected to the proximal end 14 of the probe. The TDR sensor is configured to generate electrical pulses through the waveguides 42 and receive signals indicating changes in impedance along the electrical waveguides, including those induced by conductive impedance element 50. Impedance element 50 may be a conductive ring encircling probe segment 40 that is in contact with electrical waveguides and through which probe body 40 moves longitudinally while conductive ring remains stationary. As the probe segment moves into or retracts from a lumen (e.g., lumen 35 of FIG. 2A), the time of travel of an impedance echo relative to element 50 correspondingly changes. The time change is reflected in signals received by the TDR sensor and is used by system 28 to calculate relative longitudinal distances traveled by probe body 40 within the lumen. These distances may be stored in computer memory and related in computer memory to the cross-sectional (slice) representations of the lumen described above.

FIG. 2A is an illustrative side perspective diagram of an ultrasound catheter probe placed within a lumen according to some embodiments. FIG. 2B is across-sectional perspective diagram of the ultrasound catheter probe of FIG. 2A. Catheter probe 10 is shown inserted into a lumen 35. Connected computer system 36 is programmed to cause transducers 18 to generate pulses 45 where each of the pulses is incident on different portions of lumen 35. In response to echoes from lumen walls 35, transducers 18 generate electromagnetic signals respective to the pulses that reflect (i.e., echo) back from media and portions of the lumen walls 35 adjacent probe 10. These electromagnetic signals are then processed by a signal processor and computer system 36. In some embodiments, an envelope signal associated with the activating pulse is detected and distinguished within the return signals to identify a transition between media and/or structural features. Based on the distinction, a distance measurement may be calculated between the transducer/probe and the transition location.

Other pulses may be similarly delivered/echoed using other transducers 18. In some embodiments, these pulses may be delivered simultaneously or at different times. Along with identifying and associating the signals with respective transducers, the computer system 36 is programmed to analyze the signals and calculate a radial distance measurement between each transducer 18 and lumen 35. This may be done, for example, by utilizing time-of-flight information of the echo signals and previously determined/differentiated signatures representative of lumen wall 35 and a particular medium (e.g., blood) between the transducer 18 and lumen walls 35. Exemplary systems and methods for making such calculations are described, for example, in U.S. Pat. No. 10,231,701 filed Mar. 14, 2014 (the '701 Patent), the entire contents of which is herein incorporated by reference.

Based on distance calculations, the shape and dimensions of the lumen may be estimated by further utilizing information including the dimensions of probe 10 and applying interpolation and/or other mathematical fitting techniques. For example, the relative positions of points about lumen walls at 35A and/or 35B may first be calculated and a curve fitting algorithm (e.g., spline interpolation) is applied to generate a two-dimensional slice representation of the lumen based on signals from the walls at 35A and 35B. As described in the '701 Patent, multiple slices can be calculated by taking sets of ultrasound readings along the longitudinal extent of the lumen and combining them to generate a two-dimensional representation with depth or a two and a half-dimensional representation.

The curve fitting may be performed by combining the points of the lumen walls at different times and positions. In some embodiments, each set of points is analyzed to determine their relative positions with respect to each other (i.e., with respect to the overall lumen 35). In some embodiments, a centroid of each set of points is determined and the centroids are used as a common point that corresponds to both sets. After determining the centroid (or other correlation), the points are set and combined according to the common origin and a curve is fitted (e.g., by splines) using the combined points to determine a refined shape/size of the lumen. Multiple sets of points across the lumen can be obtained and similarly combined and fitted.

These sets of points may be related (e.g., in computer memory storage) to longitudinal distance measurements using a TDR sensor (e.g., of acquisition device 36 of FIG. 1). The cross-sections may be further fitted with respect to each other based on the longitudinal distance measurements and used to generate a three-dimensional representation of the lumen.

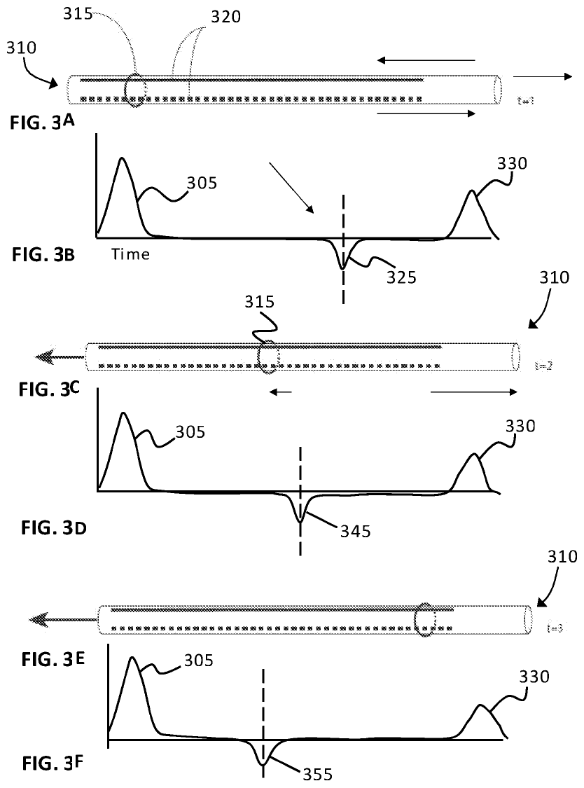

FIGS. 3A, 3C, and 3E are illustrative side perspective views of a probe body integrated with TDR waveguides according to some embodiments. FIGS. 3B, 3D, and 3F are illustrative charts of TDR signals detected from the TDR waveguides of FIGS. 3A, 3C, and 3E, respectively, according to some embodiments. A probe body 310 (e.g., similar to probe body 40 of FIG. 1) includes a conductive ring 315 which is arranged to slide longitudinally along probe body 310. One or more conductive electrical waveguides 320 extend along probe body 310 (e.g., along probe body 310's outer surface) so that conductive ring 315 comes into contact with waveguides 320 while conductive ring 315 moves longitudinally. At a proximal end of probe body 310, a TDR sensor (not shown) is configured to measure impedance signals across electrical waveguides 320 in response to electrical and/or electro-magnetic pulses generated by the TDR sensor.

In some embodiments, the one or more conductive electrical waveguides 320 include two electrical waveguides where ring 315 completes an electrical connection (or short) between the electrical waveguides. The characteristics of the connection provided by the ring 315 represent a change in impedance of signals through electrical waveguides 320. The presence and location of the connection may be reflected in an impedance peak (e.g., peak 325 of FIG. 3B) in TDR signals generated by the TDR sensor.

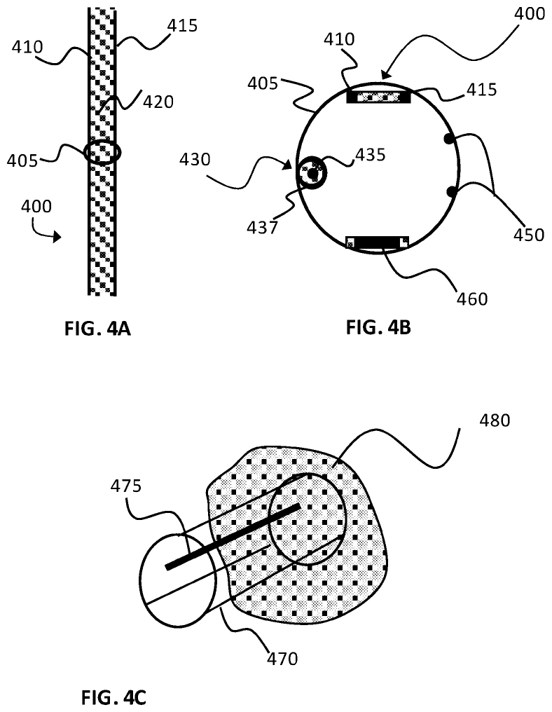

In some embodiments, the one or more conductive electrical waveguides 320 including a single waveguide that may be configured to operate as an electro-magnetic (e.g., radio frequency) waveguide (e.g., similar to a coaxial cable or conductive coaxial strip shown in FIGS. 4A and 4B) and the ring 315 (or other signal interfering element) causes an interference with signals traveling through the waveguide. A pulse by a TDR sensor through the waveguide will reflect the interference (e.g., a peak 325 shown in FIG. 3B) and longitudinal distance between the TDR sensor and interfering element is calculated. In some embodiments, a frequency domain reflectometry (FDR) sensor is utilized by sweeping across a range of frequencies to detect impedances and determine a longitudinal distance.

FIG. 3B illustrates amplitude measurements of signals received by a TDR sensor over time. An initial peak 305 illustrates a signal associated with the initial pulse generated by the TDR sensor. A second impedance peak (or minimum) 325 illustrates an impedance signal or echo responsive to a change in impedance caused by conductive ring 315 along electrical waveguides 320. The timing of impedance peak 325 with respect to when the initial pulse was generated will correlate with the distance along the conductive path between the TDR sensor and conductive ring 315. A “time of flight” determination is made and stored with respect to the first position of conductive ring 315 shown in FIG. 3A.

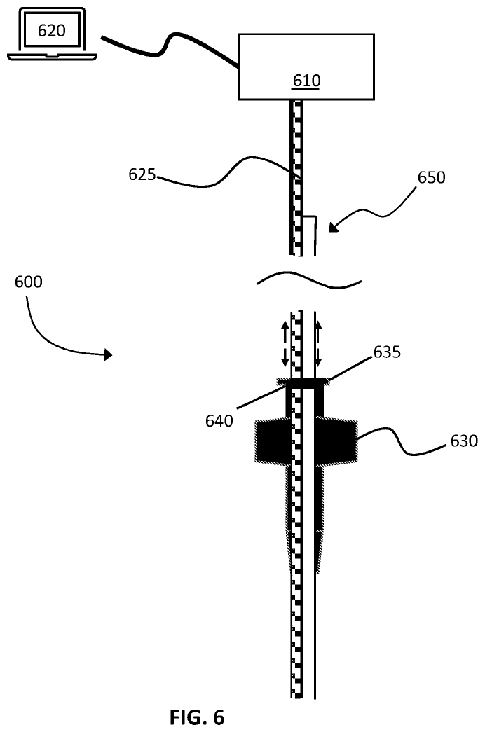

As shown in FIG. 3C, conductive ring 315 is at a second position after probe body 310 has been moved (or inserted) further (forward) into a structure (e.g., a patient). Conductive ring 315 is held stationary with respect to probe body 310 as probe body 310 is moved forward. The conductive path thereby becomes shorter between conductive ring 315 and the TDR sensor as compared to the conductive path to the ring 315's original position in FIG. 3A. In some embodiments (e.g., as shown in FIG. 6), conductive ring 315 is held in place manually (e.g., by hand) by an operator (e.g., a clinician) as the probe body 310 is driven forward or pulled backwards within a patient's body.

After the conductive path between the TDR sensor and ring 315 is shortened, the resulting response shown in FIG. 3D reflects a correspondingly shorter echo time for the impedance echo peak 345 compared to the impedance echo peak 325. The change in echo time between signal peaks 325 and 345 is used to determine the longitudinal movement of ring 315. In some embodiments, the determination is performed by a system (e.g., system 28) configured with a calibration factor based on comparing previous independent measurements of longitudinal movement and echo signal times.

In FIG. 3E, probe body 310 has been further moved forward while ring 315 has been held stationary. Another TDR measurement illustrated by FIG. 3F reflects a shorter timed echo signal 355. Based on the timing of echo signal 355, the longitudinal movement of probe body 310 is calculated with respect to previous positions illustrated in FIGS. 3A and 3C.

FIG. 4A is an illustrative cross-sectional view of a flat cable 400 for use with a TDR system and an ultrasound probe according to some embodiments. Flat cable 400 is integrated with a probe body (e.g., probe body 40 of FIG. 1) and connected with a TDR sensor to measure changes in longitudinal positions of the probe body such as further described herein. Flat cable 400 includes an electrical signal conductor 410 and a ground conductor 415, between which is an electrically insulating material 420. Flat cable 400 may be constructed of thin flat strips of conductive material (e.g., metal, conductive polymers) for use as the signal conductor 410 and ground conductor 415 and a thin insulating material constructed of non-conductive material (e.g., polyamide).

When a conductive impedance element 405 (e.g., impedance element 50 of FIG. 1) comes into contact (e.g., direct or in substantially close proximity) with waveguide 400, a TDR signal transmitted through flat cable 400 is impeded and causes an echo impedance signal (e.g., similar to what is shown in FIG. 3B) to be returned. As described further herein, the echo signal (e.g., impedance signal) may be used to calculate changes in the longitudinal position of the probe body as the probe body moves through the conductive impedance element. In some embodiments, the TDR pulse and echo signals are transmitted as electromagnetic (e.g., radiofrequency) signals.

FIG. 4B is an illustrative perspective view of different types of waveguide arrangements for use with a TDR system and an ultrasound probe according to some embodiments. The flat cable 400 is shown from a front cross-sectional view surrounded by and in contact with a conductive element 405 as it would be when integrated with a probe (e.g., probe 10 of FIG. 1). In some embodiments, multiple conductive wires 450 are used to transmit TDR pulses as further described herein so that the conductive element 405 creates a “short” between wires 450 and causes an impedance echo signal to be generated that is representative of the movement/position of the probe within a structure.

In some embodiments, a coaxial cable 430 is used to transmit TDR pulses and includes an inner conductive signal carrier 435 and outer conductive layer 437. Where conductive element 405 is in contact with cable 430, an impedance response signal is generated and can be used to determine the movement and position of the probe. In some embodiments, a flat cable 460 includes a single conductive flat wire that is used to transmit TDR pulses. When a TDR pulse is transmitted through cable 460, conductive element causes a responsive signal (e.g., impedance or RF signal) to be echoed back where it is in direct or close contact with cable 460.

FIG. 4C is an illustrative perspective view of a probe segment utilizing a conductive fluid medium as an impedance element for TDR implementation according to some embodiments. A probe body 470 is integrated with one or more conductive waveguides 475 (e.g., such as shown in FIGS. 4A and 4B). Probe body 470 is integrated with a probe (e.g., probe 10 of FIG. 1) and is shown inserted into a conductive fluid medium 480 (e.g., the blood of a patient's blood vessel) during an intravascular procedure. Where conductive waveguides 475 come into contact with conductive medium 480, an echo signal is generated in response to a TDR pulse and used to track the longitudinal movement of probe body 470 in the medium 480 in similar fashion as to when a conductive element (e.g., conductive element 405) is utilized.

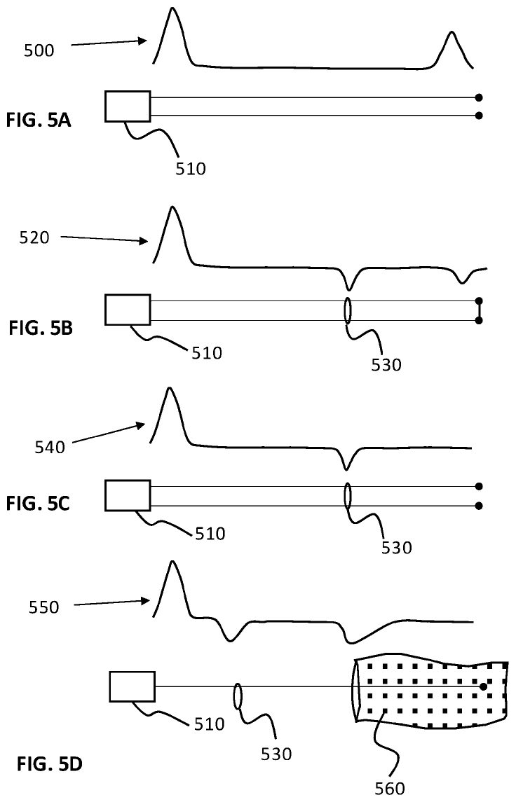

FIG. 5A is an illustrative diagram of a TDR open circuit arrangement and chart of a TDR signal. A TDR sensor 510 is connected with two cables (e.g., signal and ground) that may be integrated with an ultrasound probe system (e.g., system 28 of FIG. 1). A TDR signal chart 500 illustrates a signal generated and detected through TDR sensor 510. An open circuit implementation will typically cause a first and second peak representing the beginning and terminating ends of signal travel.

FIG. 5B is an illustrative diagram of a TDR parallel impedance arrangement and TDR signal chart 520 of a TDR signal according to some embodiments. TDR sensor 510 is connected to a closed-circuit loop about which an impedance element 530 is able to move longitudinally (e.g., conductive ring 315 of FIGS. 3A-3E). In response to a TDR signal, a peak (or minimum) is generated that reflects the position of the impedance element 530 along the circuit/probe such as further described herein.

FIG. 5C is an illustrative diagram of a TDR short circuit arrangement and TDR sensor chart 540 of a TDR signal according to some embodiments. Conductive impedance element 530 causes an impedance signal corresponding to the location of the short (i.e., the position of impedance element 530).

FIG. 5D is an illustrative diagram of a single-cable TDR arrangement presented with different types of impedance and a TDR signal chart 550 of corresponding TDR signals according to some embodiments. A signal (e.g., a radiofrequency signal) can be generated by TDR sensor 510 so that it travels along a conductive waveguide (e.g., cable/wire). In some embodiments, the signal is generated at frequencies of between about ten to fifteen megahertz (MHz) or other frequencies determined to be responsive to the particular type of impedance being measured. A responsive signal will indicate the relative location of an impedance change (e.g., from impedance element 530). In some embodiments, the impedance change of a conductive medium 560 (e.g., fluid including water and blood) into which the cable (or cables) is inserted is identified and used to calculate the relative distance of travel (e.g., within a blood vessel) of the probe with which the TDR system it is integrated.

FIG. 6 is an illustrative view of a TDR system 600 integrated with a probe body 650 according to some embodiments. TDR system 600 includes a TDR sensor 610 that is connected to and controlled by a computing device 620. One or more wires/cables 625 are connected to TDR sensor 610 through which TDR sensor transmits TDR electrical signals and receives feedback signals (e.g., as shown in FIGS. 5A-5D). Probe body 650 is slidably movable within a trocar 630 that may be positioned at an insertion point of a patient and guide the longitudinal movement of probe body 650 therethrough. Trocar 630 includes a locking mechanism 635 that prevents the longitudinal movement of probe body 650 through it when in it's in a locked position. For example, the locking mechanism may include a rotatable cap or knob that, when turned in a particular direction, compresses a portion of the trocar 630 on the probe body and decompresses and releases the probe body 650 when turned in the opposite direction.

In some embodiments, an impedance element 640 (e.g., a conductive ring element) is integrated with trocar 630 and arranged to contact the one or more wires/cables 625 of probe body 620. In response to signals from TDR sensor 610, impedance element 640 causes responsive TDR signals to be received by TDR sensor 610 that indicate the relative longitudinal distance between TDR sensor 610 and trocar 630. As the probe body 620 is moved within trocar 630 (and in and out of a patient), the relative movement of the probe body is tracked.

Computing device 620 is programmed and configured to cause TDR sensor 610 to generate TDR signals (e.g., electrical/electromagnetic signals) that travel through the one or more wires/cables 625. These signals and their characteristics may be configurable by an operator of computing device 620 (e.g., through a graphical user interface) and may include, for example, the magnitude (e.g., maximum voltage), frequency, waveform, and/or other characteristics. Computing device 620 may also receive responsive TDR signals and convert/translate them into relative longitudinal travel distances/positions of probe body 650. Computing device 620 may be programmed to cause a graphical display to show the calculated position of probe body 650 (e.g., including its distal end) within a structure (e.g., patient body). In some embodiments, the calculated position is co-registered with images (e.g., CT, MRI, ultrasound) of the structure in which the probe is positioned and represented in display renderings of these images. In some embodiments, the calculated positions and co-registered images are utilized to guide the positioning of probe body 650 within a structure (e.g., blood vessel) in real-time.

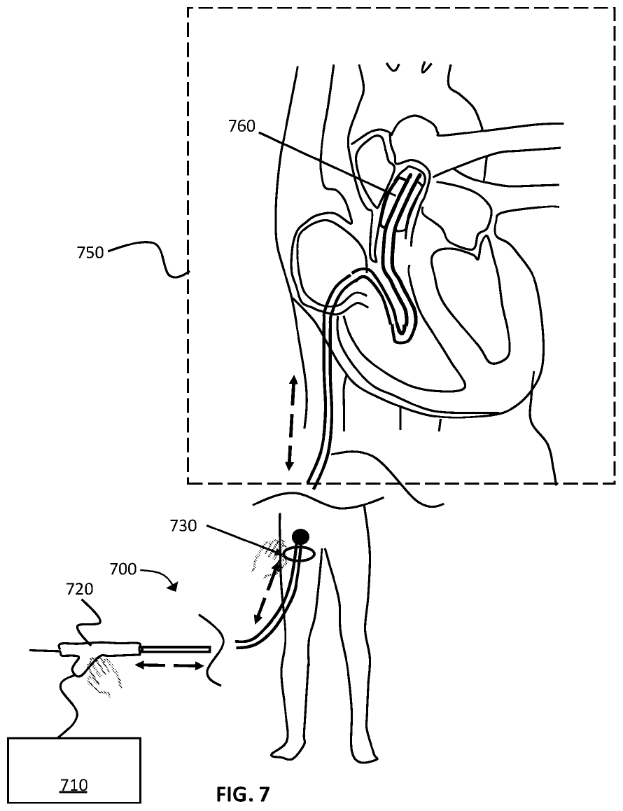

FIG. 7 is an illustrative view of an ultrasound probe 700 with a TDR sensing system inserted into a heart 750 according to some embodiments. Ultrasound probe 700 includes one or more TDR cable/wire waveguides (not shown) connected to a TDR sensor 710 and a conductive impedance element 730 in contact with and held stationary with respect to the longitudinal movement of the TDR cable/wires and probe 700. Sensor 710 is configured to generate and obtain TDR signals such as described further herein. A handle 720 located at the distal end of probe 700 may be used to guide the longitudinal movement of probe 700 in heart 750. A computing device (e.g., similar to computing device 620 of FIG. 6) is connected to TDR sensor 710 and configured to compute/translate TDR signals into longitudinal position measurements of probe 700 within heart 750.

Probe 700 includes one or more devices (e.g., device 43 of FIG. 1) at its distal end that may be used to perform measurements of and/or treat areas of heart 750. Probe 700 may include, for example, a lumen-expanding balloon (e.g., angioplasty balloon), obstruction crossing tool, and/or an ultrasound measurement transducers (e.g., as described with respect to FIGS. 1, 2A-2B). Measurements from probe 700 (e.g., of lumen dimensions) may be co-registered with positional information obtained from TDR sensor 710 and other imaging modalities and used to guide the treatment of heart 750 (e.g., expanding/unblocking blood vessels).

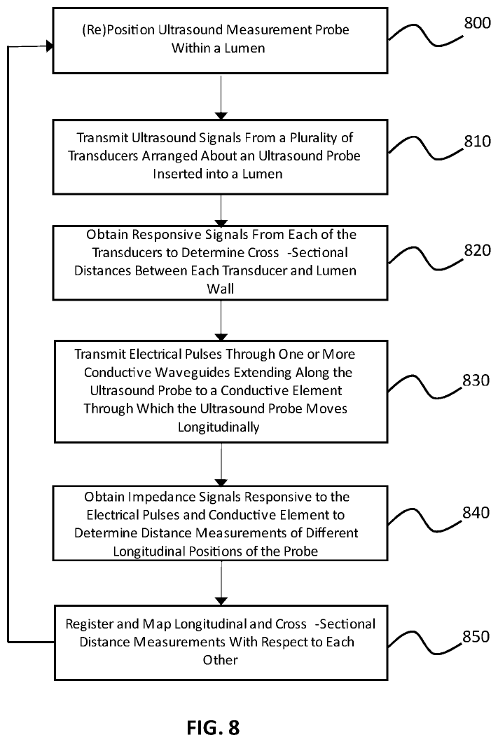

FIG. 8 is an illustrative flow chart of a process for performing ultrasound measurements with longitudinal position measurements according to some embodiments. At block 800, an ultrasound probe with a plurality of transducers (e.g., as described with respect to FIGS. 1, 2A-2B) is positioned within a lumen (e.g., as part of a percutaneous coronary procedure). At block 810, the probe transmits ultrasound signals to surrounding structure (e.g., a blood vessel walls). At block 820, ultrasound signals obtained in response to the signals transmitted at block 810 are used to calculate radial distances between the probe and structure (e.g., as described in the '701 patent). Based on the distance calculations, a cross-sectional shape and dimensions (e.g., diameters, area) of the cross-section are determined.

At block 830, a TDR sensor integrated into the ultrasound probe transmits electrical TDR signals through one or more conductive waveguides extending along the probe. The probe includes a conductive impedance element (e.g., conductive impedance element 50 of FIG. 1) positioned in contact with the conductive waveguides and causes reflective signals to be transmitted/echoed back to the TDR sensor in response to the initially transmitted TDR signals. At block 840, the TDR sensor receives the reflective signals, which are then converted into longitudinal distance measurements such as further described herein. The distance measurements are used to track the relative longitudinal position of the probe within the lumen.

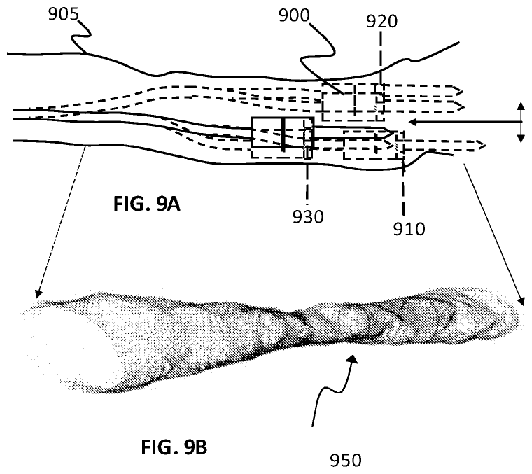

At block 850, the longitudinal position of the probe calculated at block 840 is related/co-registered (e.g., in computer memory) with the corresponding cross-sectional measurements determined at block 820. The probe may be repositioned (e.g., inserted further or pulled back) at block 800, after which additional cross-sectional and longitudinal position measurements are performed. The multiple positions and related co-registered cross-sectional measurements may be used to generate a mapping of the lumen over a span of the longitudinal positions (e.g., as shown and described with respect to FIGS. 9A and 9B).

FIG. 9A is an illustrative side perspective view of an ultrasound measurement probe 900 with a plurality of ultrasound transducers and a TDR system (e.g., such as described with respect to FIG. 1) longitudinally actuated within a vessel structure according to some embodiments. FIG. 9B is an illustrative mapping of the vessel structure of FIG. 9A based on ultrasound and TDR measurements obtained according to some embodiments. The distal end of a probe 900 is shown at multiple longitudinal positions 910, 920, and 930 within a blood vessel 905. At each of the multiple positions, cross-sectional distance measurements (e.g., shape, dimensions) are obtained using the ultrasound transducers. Longitudinal TDR measurements are also obtained and associated with the cross-sectional measurements (such as described with respect to FIG. 8). Based on the cross-sectional and longitudinal position measurements, a computer-generated three-dimensional mapping 950 of the lumen is generated and illustrated in FIG. 9B.

The processes described herein (e.g., the processes of FIG. 8) are not limited to use with the hardware shown and described herein. They may find applicability in any computing or processing environment and with any type of machine or set of machines that is capable of running a computer program. The processes described herein may be implemented in hardware, software, or a combination of the two. The processes described herein may be implemented in computer programs executed on programmable computers/machines that each includes a processor, a non-transitory machine-readable medium or other article of manufacture that is readable by the processor (including volatile and non-volatile memory and/or storage elements), at least one input device, and one or more output devices. Program code may be applied to data entered using an input device to perform any of the processes described herein and to generate output information.

The processing blocks (for example, in the processes of FIG. 8) associated with implementing the system may be performed by one or more programmable processors executing one or more computer programs to perform the functions of the system. All or part of the system may be implemented as, special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device, and/or a logic gate.

The processes described herein are not limited to the specific examples described. For example, the process of FIG. 8 is not limited to the specific processing orders illustrated. Rather, any of the processing blocks of FIG. 8 may be re-ordered, combined or removed, performed in parallel or in serial, as necessary, to achieve the results set forth above.

Elements of different embodiments described herein may be combined to form other embodiments not specifically set forth above. Various elements, which are described in the context of a single embodiment, may also be provided separately or in any suitable sub combination. Other embodiments not specifically described herein are also within the scope of the following claims.

1. A method of ultrasound measuring, the method comprising:
longitudinally actuating an ultrasound probe between a plurality of positions within the structure;
while the ultrasound probe is at each of the plurality of positions:
transmitting ultrasound signals from at least one ultrasound transducer of the ultrasound probe toward a structure;
obtaining signals responsive to the transmitted ultrasound signals and calculate distance measurements between the ultrasound probe and the structure based on each of the responsive signals;
transmitting electrical pulses through one or more conductive waveguides extending between a proximate end of the ultrasound probe and a portion of the probe distal to the proximate end;
obtaining reflective signals responsive to the electrical pulses, the reflective signals representing a change in characteristics of the transmitted electrical pulses as they transmit through a particular longitudinal position of the one or more conductive waveguides; and
calculating one or more longitudinal position measurements of the ultrasound probe based on the reflective signals responsive to the electrical pulses.

2. The method of claim 1, wherein calculating the longitudinal position measurements is based on analyzing time domain reflectometry (TDR) waveforms within the reflective signals.

3. The method of claim 2, wherein the TDR waveforms are generated in response to an impedance change through the one or more conductive waveguides created by a conductive element positioned along the ultrasound probe, wherein the conductive element is arranged to remain stationary as the ultrasound probe is longitudinally actuated.

4. The method of claim 3, wherein the conductive element comprises a movably slidable ring arranged about the ultrasound probe.

5. The method of claim 3, wherein the conductive element is integrated within a trocar through which the ultrasound probe is arranged to pass, the trocar configured with a mechanism for locking the trocar in place to the probe.

6. The method of claim 3, wherein the conductive element comprises a fluid media in the structure within which the ultrasound probe is longitudinally actuated.

7. The method of claim 6, wherein the fluid media comprises blood.

8. The method of claim 2, wherein calculating the longitudinal position measurements is based on electrical pulses transmitted through one conductive waveguide.

9. The method of claim 2, wherein calculating the longitudinal position measurements is based on analyzing time domain reflectometry (TDR) waveforms within radio frequency waves.

10. The method of claim 1 wherein the electrical pulses comprise a wavelength range of between about ten and fifteen megahertz.

11. The method of claim 1, wherein a single waveguide is used and reflected energy is analyzed.

12. The method of claim 1, wherein multiple waveguides are used and transmitted energy is analyzed.

13. The method of claim 1, wherein, based on the calculated distance measurements between the ultrasound probe and the structure, determining a plurality of cross-sectional shapes of the structure and, based on the one or more longitudinal distance measurements, determining a longitudinal distance between the cross sectional shapes.

14. The method of claim 13, further comprising determining one or more longitudinal shapes of the structure based on the plurality of cross-sectional shapes and one or more longitudinal distances.

15. The method of claim 14, further comprising determining a three-dimensional shape of the structure based on the determined longitudinal shapes and the plurality of cross-sectional shapes.

16. The method of claim 13, wherein the plurality of cross-sectional shapes are registered in computer memory as relative longitudinal positions within the structure based on the one or more longitudinal measurements.

17. An ultrasound system for measuring dimensions of a structure, the system comprising:
a flexible body elongated along a longitudinal axis and assembled for insertion into the structure;
at least one ultrasound transducer arranged on the flexible body;
one or more conductive waveguides extending between a proximate end of the flexible body and a portion of the flexible body distal to the proximate end;
one or more processors programmed and configured to:
transmit ultrasound signals from the at least one ultrasound transducer;
obtain signals responsive to the transmitted ultrasound signals and calculating a distance measurement between the flexible body and the structure based on each of the responsive signals;
transmit electrical pulses through the one or more conductive waveguides;
obtain reflective signals responsive to the electrical pulses, the reflective signals representing a change in characteristics of the transmitted electrical pulses as they transmit through a particular longitudinal position of the one or more conductive waveguides; and
calculate one or more longitudinal position measurements of the flexible body based on the reflective signals responsive to the electrical pulses.

18. The system of claim 17, wherein calculating the one or more longitudinal position measurements is based on analyzing time domain reflectometry (TDR) waveforms within the reflective signals.

19. The system of claim 18, wherein the TDR waveforms are generated in response to an impedance change through the one or more conductive waveguides created by a conductive element positioned along the flexible body, wherein the conductive element is arranged to remain stationary as the flexible body is longitudinally actuated.

20. The system of claim 19, wherein the conductive element comprises a movably slidable ring arranged about the flexible body.
